(19) United States
(12) Patent Application Publication
(10) Pub. No.: US 2024/0221188 A1
(43) Pub. Date: Jul. 4, 2024

(54) 3D Contagion Mapping Through Visual Exhale Monitoring

(71) Applicant: The Regents of the University of Colorado, a body corporate, Denver, CO (US)

(72) Inventors: Min-Hyung Choi, Superior, CO (US); Shane Transue, Arvada, CO (US)

(21) Appl. No.: 18/402,003

(22) Filed: Jan. 2, 2024

Related U.S. Application Data
(60) Provisional application No. 63/478,077, filed on Dec. 30, 2022.

(51) Int. Cl. G06T 7/292; G16H 50/50

(52) U.S. Cl. CPC G06T 7/292; G16H 50/50; G06T 2207/10016; G06T 2207/10028; G06T 2207/20024

(57) ABSTRACT

Systems and methods for contagion mapping are disclosed herein. An implementation of the contagion modeling system based on a thermal depth video capture is disclosed. The modelling system includes two CO2 thermal depth imaging cameras connected to a processor. The CO2 thermal depth imaging cameras are configured so that they have fields of view within a modeling area. The modeling area includes a stationary object, a moving object and a gaseous fluid source, which is a contagion source.

### RELATED APPLICATIONS

This application hereby claims the benefit of and priority to U.S. Patent Application No. 63/478,077, titled “3D CONTAGION MAPPING THROUGH VISUAL EXHALE MONITORING,” filed Dec. 30, 2022, which is hereby incorporated by reference in its entirety.

### TECHNICAL FIELD

Aspects of the disclosure are related to the fields of healthcare and disease prevention and, more particularly, to accurately modelling potential spread of contagions in real-life scenarios.

### BACKGROUND

With increased attention to the spread of contagions, the ability to accurately map the potential spread of contagion increases in importance. While various models have been created, and can be created using existing floorplans and layouts, it is difficult to accurately measure how the contagion spreads through the air and on various surfaces.

### OVERVIEW

A contagion modeling system based on a thermal depth video capture is disclosed. The modelling system includes two CO2 thermal depth imaging cameras connected to a processor. The CO2 thermal depth imaging cameras are configured so that they have fields of view within a modeling area. The modeling area includes a stationary object, a moving object and a gaseous fluid source, which is a contagion source.

A method of modeling the spread of a contagion is also disclosed. The method includes receiving image data from a CO2 filtered thermal camera. The image data contains a gaseous flow contagion source, a stationary object and a moving object. The method also includes translating the image data from the first CO2 thermal depth imaging camera into a 3D density-flow representation. Additionally, the method includes processing the 3D density-flow representation with at least the stationary object and the moving object to produce a predicted contagion flow pattern.

A method of modeling a system to predict contagion dispersion is also disclosed. The method includes acquiring a thermal depth video stream. The method includes identifying two gaseous fluid sources in the thermal depth video stream and converting the two gaseous fluid sources in the thermal depth video stream into two fluid dispersion models. The method determines that the first fluid dispersion model may interact with the second fluid dispersion model and a first stationary object, resulting in a density of the first fluid that is above a threshold value in a location. The method additionally includes determining a recommended layout change in response to the determination that the density of the first fluid is above the threshold value in the first location and indicating the recommended layout change.

### BRIEF DESCRIPTION OF THE DRAWINGS

Many aspects of the disclosure may be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIG. 1 illustrates a camera in an example implementation.

FIG. 2 illustrates a training process in an implementation.

FIG. 3 illustrates a modeling system.

FIG. 4 illustrates a device network in an implementation.

FIG. 5 illustrates an analysis process in an implementation.

FIG. 6 illustrates a modeling system.

FIG. 7 illustrates a modelling process in an implementation.

FIG. 8 illustrates an operational sequence in an implementation.

FIG. 9 illustrates a computing system suitable for implementing the various operational environments, architectures, processes, scenarios, and sequences discussed below with respect to the Figures.

### DETAILED DESCRIPTION

Technology disclosed herein relates to systems and methods for modelling the spread of contagions in real and hypothetical spaces. In particular, a method is presented of quantifying gaseous fluid flow and incorporating it into design models. The models can be analyzed and changes to reduce the likelihood of spreading contagions can be presented and analyzed.

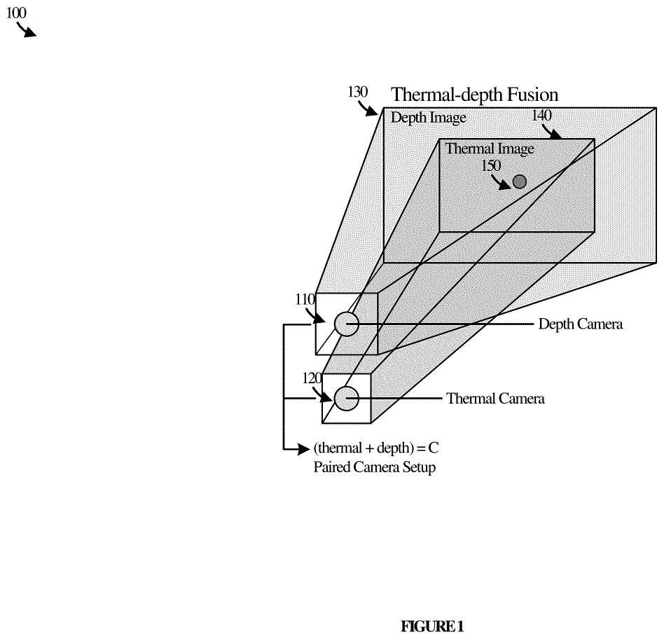

In an implementation, gaseous contagion spread can be analyzed through photographic data. In FIG. 1, a system 100 is shown in accordance with an implementation. Depth camera 110 can be a camera capable of sensing depth. This could be, for example, a stereo vision camera, a time-of-flight camera, or a structured light camera, for example. Thermal camera 120 can be, for example, an infrared camera that is capable of creating an image based on the amount of heat in an object. The images from thermal camera 120 can be filtered to particular wavelengths in order to more closely define the images that are captured. Depth camera 110 and thermal camera 120 each have a field of view. The fields of view 130 and 140 shown in FIG. 1 differ slightly in size, but one of ordinary skill in the art should recognize that the fields of view 130 and 140 could also directly coincide.

Depth camera 110 and thermal camera 120 can be physically separate units, or they can be combined in one apparatus. Similarly, the images produced by depth camera 110 and thermal camera 120 can be separate, or a combined camera may internally process each of the images and output a combined thermal-depth image. In an implementation, depth camera 110 and thermal camera 120 produce images repeatedly, such as video images, such that a series of time-based images is produced.

Object 150 is shown in the thermal field of view 140 and the depth field of view 130. Object 150 can be any type of gaseous contagion, including, for example, an exhaled breath from an infected person. In an implementation, object 150 is captured in a set of time-based thermal images by thermal camera 120, and a corresponding set of time-based depth images by depth camera 110. These images can be combined to create a time-based set of 3D thermal images.

In an implementation, object 150 is an individual, particularly, an individual's head. Thermal camera 120 and depth camera 110 captures time-sequenced images of the individual's head as the individual breathed. The thermal images are filtered such that they display exhaled CO2 from the individual's mouth and nose. The sets of images from thermal camera 120 and depth camera 110 are combined to create a series of 3D thermal images that show CO2 exhaled by the individual.

This series of 3D thermal images can be converted into vectors representing the flow of CO2 in the exhalation. The images also contain magnitude information which can be associated with the flow vectors. In an implementation, this magnitude and flow information can be combined to create a model of the flow of CO2 being exhaled by the individual.

In an implementation, this model of flow can be correlated with actual known flow metrics. Many currently used pulmonary diagnostic tests use known metrics. By correlating the exhaled CO2 flow model with standard metrics, diagnostics can be enhanced. In an implementation, breath activity can be simultaneously measured by a currently available flow measurement, such as spirometry, and by the thermal camera 120 and depth camera 110 currently being described. After processing, the CO2 flow model of exhaled CO2 can then be calibrated with the other measured (e.g., spirometry) data. This could be a simple correlation, or it could be a much more complex machine learning process that incorporates the flow vector and magnitude data calculated from the thermal and depth images in order to create predictions of flow that correspond closely to the other measured (e.g., spirometry) data. The result of this calibration is that the data produced by the thermal camera 120 and depth camera 120 can be converted into estimations or predictions of flow metrics for the individual being photographed by the cameras. These flow metrics could include, for example, volume, volumetric flow, and velocity, among others.

Similar methods could be used to calibrate thermal and depth images to flow metrics for other sources of contagion. It should be understood that various flow measurements, wavelength filters, etc., could be used to complete this calibration.

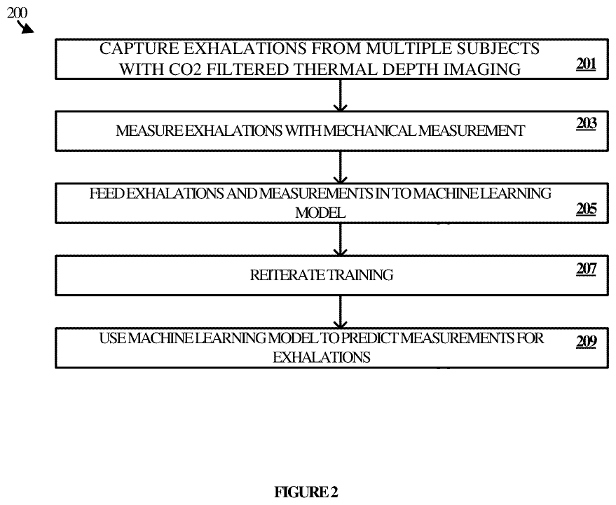

FIG. 2 shows a method of calibrating a CO2 flow model from thermal and depth images to measured flow metrics. In step 201, Exhalations are captured from multiple subjects. As discussed above, the thermal camera 120 and depth camera 110 can be used to record images of an individual exhaling. This same process can be used for multiple subjects, creating a collection of time-sequenced images corresponding to various subjects' exhalations. These time-sequenced images can be converted into a collection of CO2 flow models. In an implementation, each subject is recorded for several exhalations in order to create a large sample size.

While the exhalations are being recorded, they are also measured directly with a spirometer in step 203. Various adjustments can be made in order to account for changes in the signal due to the presence of the spirometer. For example, the air can be heated such that is exits the spirometer at or near the temperature it would have been at the exit from the subject's mouth or nose. Following these measurements, a collection of CO2 flow models is stored, together with corresponding spirometry measurements.

In step 205, this data, or some of this data, is fed into a machine learning model. The machine learning model can be configured to correlate the flow vectors and magnitude information from the thermal and depth images with the velocity and volume measurements from the spirometer. The samples of data fed into the machine learning model can be selected to ensure that high quality data is used. In an implementation, the machine learning model can be used to evaluate breathing from multiple different subjects, with many breaths from each subject. This can allow for a correlation that provides reasonable accuracy across a large segment of the population. In another implementation, the machine learning model can be used to evaluate breathing over many breaths from a single individual. In this way, higher accuracy can be achieved for that particular individual. Step 207 shows that the machine learning can be iterated as many times as preferred. In some cases, a machine learning model that has already been put to use can be further refined by adding more patients and/or breath cycles as input to the model.

In step 209, the machine learning model can be used to process image data from depth camera 110 and thermal camera 120 to estimate or predict flow metrics for a patient in fields of view 130 and 140. Thus, by creating images for a patient, a prediction can be made for flow, volume and/or velocity that corresponds to a similar measurement that would be made using traditional measurement, such as a spirometer. This allows for measurement of natural breathing, as the subject can be recorded in any position, and for any amount of time, allowing for relaxation. Similarly, no additional breathing obstruction needs to be added to the airflow for measurement. The prediction of flow metrics also allows for predictive capabilities. For example, given an image, or time-based set of images of a gaseous flow, the future volume and location of the as can be predicted. This allows for prediction of movement of airborne contagions.

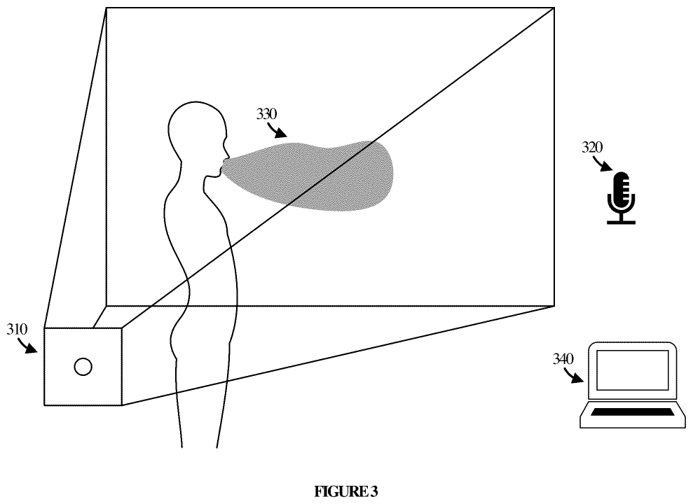

FIG. 3 shows a diagnostic system for diagnosing pulmonary conditions. Camera 310 is a combined depth/thermal camera, as discussed above. In an implementation, camera 310 is able to directly output predicted flow metrics as described above. In another implementation, camera 310 is able to output thermal and depth images which can be processed to produce flow metrics as discussed above. FIG. 3 shows a single combined thermal/depth camera 310, but distinct thermal and flow cameras could be implemented. Further the diagnostic system could use multiple cameras 310, either from similar orientations, or from differing orientations, in order to collect additional data.

Microphone 320 is shown as a single microphone but could similarly be an array of microphones. In an implementation, microphone 320 is correlated with camera 310, such that the data that is collected from camera 310 and microphone 320 is correlated. This correlation could be through the use of time stamps, file integration, or some other way.

Flow 330 is shown in the field of view of camera 310. This could be an exhalation from a human or animal subject as shown, or any other source of gaseous fluid flow. As discussed above, camera 310 is configured to produce flow data related to flow 330. This flow data can similarly be correlated with the data from microphone 320. The data from camera 310, microphone 320, and is transmitted to computer 340. Computer 340 contains a processor and memory. Computer 340 could be any type of computer, such as a personal computer, laptop, server, smart phone, specialized computer, etc. Computer 340 is configured to process the data from camera 310 and microphone 320.

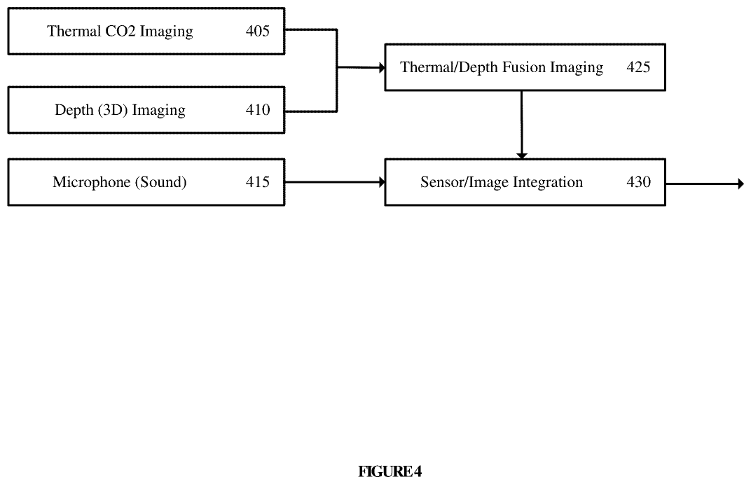

FIG. 4 illustrates an implementation of a processing progression of a diagnostic system. Element 405 represents thermal imaging. In an implementation, this imaging is done using a thermal camera which captures heated airflow. For example, a thermal camera can be used to capture breath (warm and primarily CO₂) leaving a subject. The subject could be a human subject or some other subject, such as an animal. In an implementation, this measurement could take place on a non-living subject. While an image is discussed, it should be understood that this discussion also applies to video recording or a plurality of images.

A 3D depth image is collected in element 410. This can be performed by any type of 3D or depth camera that can capture depth images. In an implementation, a depth image that will allow for interpretation of density of a gas cloud is collected. As with the thermal image discussed above, while an image is discussed, multiple images or video data can be collected in various implementations.

The thermal images and 3D depth images are combined in element 425. In an implementation, this combination is accomplished through the use of a camera that collects both thermal and 3D depth images at the same time and directly. In an implementation, element 425 results in a time-based series of images that provide a 3D representation of the exhalations of an individual.

In element 415, sound data is collected. In an implementation, this sound data is correlated to the time-based thermal and depth images of patient exhalations. The sound data can indicate breathing patterns or irregularities of the patient. In an implementation, this sound data is recorded by a microphone or a network of microphones.

In element 430, the sound data is integrated with the fused image data produced by element 425. In effect, this combination in element 430 can produce a stream of sound and image data, such as thermal depth video synchronized with sound. While FIG. 4 shows a certain order of combination to result in the sound and thermal depth video, one of ordinary skill in the art would understand that these pieces of data could be combined in many different ways to result in the described fused data. For example, a video camera configured to record thermal and depth images may combine all of the data directly in the camera, providing the thermal depth video as a direct output from the camera. Similarly, the respiratory belt data, discussed below, can also be combined with the thermal depth video data at any point in the process.

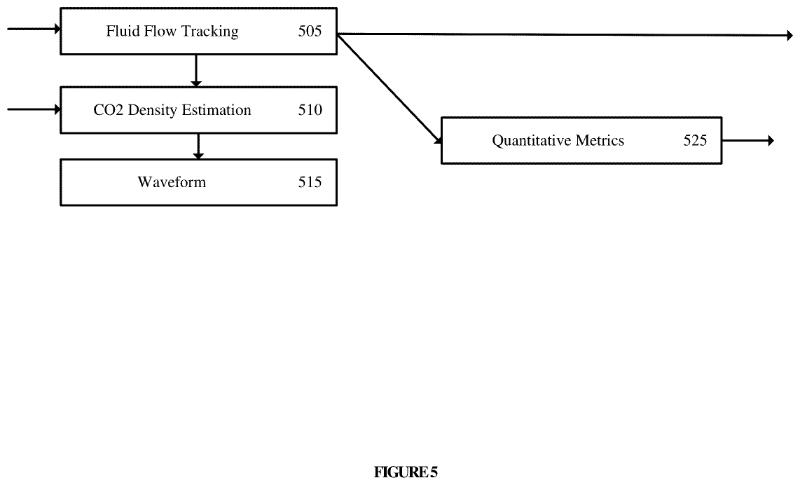

FIG. 5 illustrates an implementation of converting recorded data (such as the thermal depth image data and the sound data collected in FIG. 4) into respiratory data. In element 505, an incoming signal is processed to produce fluid flow tracking information. For example, the input signal may be an integrated thermal depth video signal as discussed above. The time-based progressive images can be converted into vector data showing movement of images within the thermal depth video. In an implementation, various filters can be used to enhance the images of CO₂ being exhaled by the subject. The movement of the CO₂ is then converted to vector data, indicating flow of exhaled CO₂ over time.

In element 510 a CO₂ density estimation is determined. In an implementation, the integrated thermal depth video signal produced in FIG. 4 is processed to determine the density of the breath cloud exhaled by a patient. The fluid flow vectors determined by element 505 may also be used as inputs for this determination. In an implementation, the thermal depth video is converted to a representation of the breaths exhaled by a patient over time. This representation can include CO₂ density information and can provide a foundation to determine actual physical characteristics of fluid exhaled.

The CO₂ density data can be converted into a waveform in element 515. In an implementation, this waveform can represent the exhalations of a patient. This data can be consistent over time, such that if a patient increases or decreases actual exhalation volume, for example, the waveform can indicate the increase or reduction. In an implementation, the initial waveform does not indicate the actual units for measurement of these characteristics. For example, while the initial waveform can indicate that the exhalation volume of a particular patient increases over time, the waveform may not be able to indicate how the exhalation volume of one patient compares to the exhalation volume of another patient in another setting. Alternatively, the waveform produced may include absolute values, allowing comparison between patients.

In an implementation, the waveform can be produced by multiple methods. For example, in one method, the input signals can be mathematically analyzed to produce a waveform. The algorithm for this mathematical analysis can be manually created for this purpose. In a second method, a machine learning algorithm can be used to analyze the input signals and produce the waveform. In some implementations, a combination of these methods may be used.

In element 520, a respiratory waveform is created that can be used as an input to element 515. This respiratory waveform can be created by an additional sensor, such as a chest movement sensor, for example. In an implementation, this respiratory waveform is an additional indicator of breathing that can be used to support the thermal depth video waveform. This respiration waveform can be used together with fluid flow vectors and CO₂ density information to create the waveform of element 515.

The data produced by elements 505-520 can then be used to produce actual absolute value data for the patients. For example, using either analytical or machine learning processes, or some combination, the visual and other data collected from the subject can be compared with measured respiration data, such as spirometry data, to develop a conversion from unitless patient respiration data to absolute respiration data. After this conversion is identified, future unitless respiration data (such as that produced from the thermal depth images described herein) can be converted to absolute respiration data without the need for spirometry measurements. In an implementation, this conversion may allow for the identification of many metrics without needing a direct spirometry measure. For example, exhale velocity, exhale volume, nose-mouth exhalation distribution and breathing effort or strength may all be measured by this method.

With the conversion identified, whether it is through analytical means or machine learning means, or some combination thereof, it can be used to convert visual data to absolute gas flow. In an implementation, this conversion can happen in real-time, with one or more sources of gas flow simultaneously. The sources can be exhalation, as discussed, or other sources of gaseous flow. In an implementation, the conversion can be used to predict contagion activity in various scenarios.

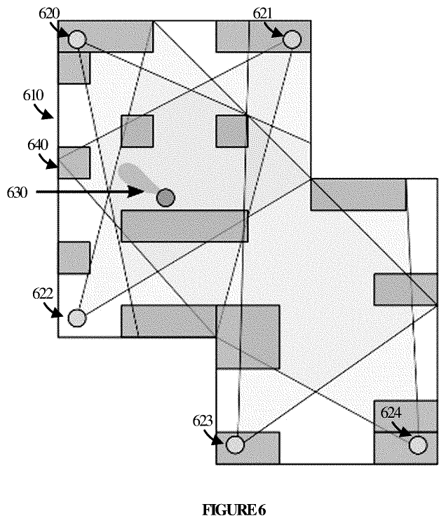

FIG. 6 illustrates an exemplary contagion model according to an implementation. Room 610 is an enclosed room. Alternatively, an open space could be modelled, and one of ordinary skill in the art would understand that various assumptions would need to be made to account for variations in air flow, external contagion sources, etc. The enclosed area, as shown in FIG. 6, may allow for a more limited set of assumptions. While FIG. 6 shows an irregularly shaped room, a square or rectangular room could also be modelled, and the process would be simplified.

Thermal depth cameras 620-624 are shown, each with a field of view directed towards a portion of room 610. While 5 cameras 620-624 are shown, any number of cameras could be utilized. For a simple square or rectangular room, a single camera may provide enough information alone. Similarly, a single camera may provide enough data to analyze a portion of a more complex room. In other implementations, more cameras may be desired.

A number of fixed objects 640 are shown throughout the room. While not all of the fixed objects are individually identified, it should be understood that many such objects are presented. These fixed objects could represent items such as desks, tables, chairs, bookcases, etc.

Contagion source 630 is also shown. Contagion source could be, for example, an individual within the room exhaling contagions. In an implementation, the contagion source is a different temperature from the surrounding room, such that the thermal depth cameras 620-624 can easily distinguish the gaseous flow of the contagion from the surroundings.

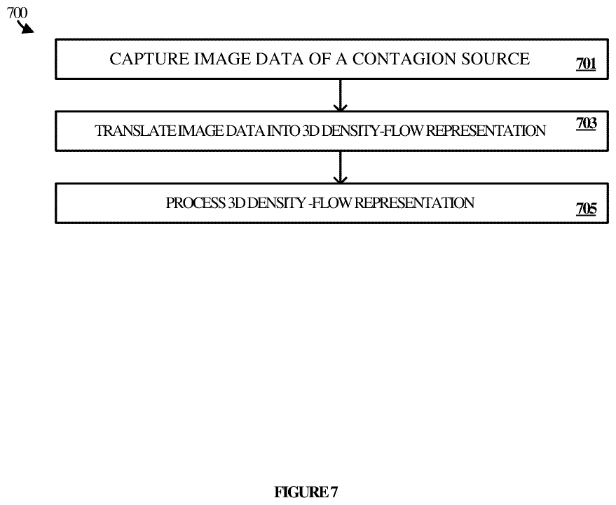

FIG. 7 illustrates a method of mapping contagions. In step 701, image data is acquired of a gaseous flow contagion. As discussed above, this can be accomplished with one or more thermal depth cameras. In an implementation, this image data can be collected as a sequential series of images of the contagions source taken by the thermal depth cameras.

The image data is translated into a density-flow representation in step 703. This translation can use magnitude and flow vector information from the sequential series of images, or it can be directly translated (either using a translation formula, or a machine learning algorithm, or some other technique).

Once the 3D density-flow representation is produced, this 3D density-flow representation can be further processed to model contagion spread. For example, in a simple room using only a single camera, and having no furniture, the model can use the 3D density flow representation to predict where the contagion “cloud” will travel, how far it will dissipate, etc. Similarly, in a more complex model with additional obstacles, the model can input the 3D density flow representation into a more complex model to determine how it will react with the other obstacles, and where the contagion will travel. One of ordinary skill in the art should also recognize that steps could be removed from this process by using a machine learning algorithm directly from image data to the contagion model, for instance. While the same principles will apply, the machine learning algorithm may not produce outcomes of the intermediate steps.

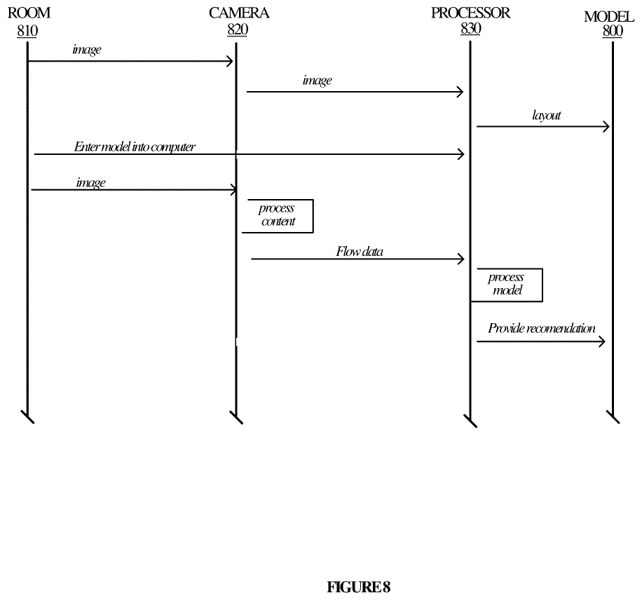

FIG. 8 shows an operational sequence of an implementation. First a model is created and entered into a computer. This entry can take a number of forms. The layout of the room, and placement of fixed objects could be entered manually through an application or design software. Alternatively, the computer could be programmed to automatically determine the room layout from image data from the thermal depth camera. In another implementation, one or more of the thermal depth cameras may be correlated with an additional camera capable of standard visible wavelength imagery. In any of these scenarios, the computer can be configured to recognize the layout, or changes to the layout, of the room and or fixed objects in the room.

According to an implementation shown in FIG. 8, an image is acquired of the room 810 by camera 820, and provided to the processor 830. This image is analyzed by processor 830 to determine a layout, which is used to create model 800, which is entered into the computer.

Next, the camera obtains image data from the room. This image data is thermal depth data which allows for prediction of gaseous flows, as discussed above. While this image acquisition is shown as a further step, the image collection to determine the room layout that was already completed could serve to accomplish both of these steps at one time. In an implementation, this image data is a time-sequenced set of images that allow for determination of flow vectors and magnitude of the contagion. This image data is processed, either at the camera, or at the processor, to determine a 3D density-flow representation. This representation is further analyzed at the processor to determine interactions with other flow values, as well as fixed and/or moving items in the room. In an implementation, the result of this analysis is a mapping that shows areas of high flow of contagions.

The processor can then further propose layout modifications that will reduce contagion hot spots. These modifications could be presented by a user through a user interface, such that the user can see changes that would come from moving fixed items, changing high traffic areas, etc. Alternatively, the computer itself could suggest alternate arrangements, such as through a machine learning algorithm that takes in thermal depth image data and produces potential changes to improve contagion risk. This process can then be repeated as many times as desired to achieve a desirable room layout.

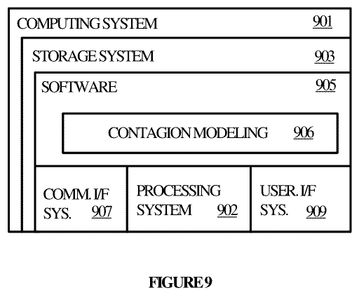

FIG. 9 illustrates computing system 901 that is representative of any system or collection of systems in which the various processes, programs, services, and scenarios disclosed herein may be implemented. Examples of computing system 901 include, but are not limited to, server computers, routers, web servers, cloud computing platforms, and data center equipment, as well as any other type of physical or virtual server machine, physical or virtual router, container, and any variation or combination thereof.

Computing system 901 may be implemented as a single apparatus, system, or device or may be implemented in a distributed manner as multiple apparatuses, systems, or devices. Computing system 901 includes, but is not limited to, processing system 902, storage system 903, software 905, communication interface system 907, and user interface system 909 (optional). Processing system 902 is operatively coupled with storage system 903, communication interface system 907, and user interface system 909.

Processing system 902 loads and executes software 905 from storage system 903. Software 905 includes and implements contagion modelling process 906, which is representative of the modelling process discussed with respect to the preceding Figures. When executed by processing system 902 to provide an operator selection process, software 905 directs processing system 902 to operate as described herein for at least the various processes, operational scenarios, and sequences discussed in the foregoing implementations. Computing system 901 may optionally include additional devices, features, or functionality not discussed for purposes of brevity.

Referring still to FIG. 9, processing system 902 may comprise a microprocessor and other circuitry that retrieves and executes software 905 from storage system 903. Processing system 902 may be implemented within a single processing device but may also be distributed across multiple processing devices or sub-systems that cooperate in executing program instructions. Examples of processing system 902 include general purpose central processing units, graphical processing units, application specific processors, and logic devices, as well as any other type of processing device, combinations, or variations thereof.

Storage system 903 may comprise any computer readable storage media readable by processing system 902 and capable of storing software 905. Storage system 903 may include volatile and nonvolatile, removable, and non-removable media implemented in any method or technology for storage of information, such as computer readable instructions, data structures, program modules, or other data. Examples of storage media include random access memory, read only memory, magnetic disks, optical disks, flash memory, virtual memory and non-virtual memory, magnetic cassettes, magnetic tape, magnetic disk storage or other magnetic storage devices, or any other suitable storage media. In no case is the computer readable storage media a propagated signal or a carrier wave.

In addition to computer readable storage media, in some implementations storage system 903 may also include computer readable communication media over which at least some of software 905 may be communicated internally or externally. Storage system 903 may be implemented as a single storage device but may also be implemented across multiple storage devices or sub-systems co-located or distributed relative to each other. Storage system 603 may comprise additional elements, such as a controller capable of communicating with processing system 902 or possibly other systems.

Software 905 (including contagion modelling process 906) may be implemented in program instructions and among other functions may, when executed by processing system 902, direct processing system 902 to operate as described with respect to the various operational scenarios, sequences, and processes illustrated herein. For example, software 905 may include program instructions for implementing a modelling process as described herein.

In particular, the program instructions may include various components or modules that cooperate or otherwise interact to carry out the various processes and operational scenarios described herein. The various components or modules may be embodied in compiled or interpreted instructions, or in some other variation or combination of instructions. The various components or modules may be executed in a synchronous or asynchronous manner, serially or in parallel, in a single threaded environment or multi-threaded, or in accordance with any other suitable execution paradigm, variation, or combination thereof. Software 905 may include additional processes, programs, or components, such as operating system software, virtualization software, or other application software. Software 905 may also comprise firmware or some other form of machine-readable processing instructions executable by processing system 902.

In general, software 905 may, when loaded into processing system 902 and executed, transform a suitable apparatus, system, or device (of which computing system 901 is representative) overall from a general-purpose computing system into a special-purpose computing system customized to provide packet redirection. Indeed, encoding software 905 on storage system 903 may transform the physical structure of storage system 903. The specific transformation of the physical structure may depend on numerous factors in different implementations of this description. Examples of such factors may include, but are not limited to, the technology used to implement the storage media of storage system 903 and whether the computer-storage media are characterized as primary or secondary storage, as well as other factors.

For example, if the computer readable storage media are implemented as semiconductor-based memory, software 905 may transform the physical state of the semiconductor memory when the program instructions are encoded therein, such as by transforming the state of transistors, capacitors, or other discrete circuit elements constituting the semiconductor memory. A similar transformation may occur with respect to magnetic or optical media. Other transformations of physical media are possible without departing from the scope of the present description, with the foregoing examples provided only to facilitate the present discussion.

Communication interface system 907 may include communication connections and devices that allow for communication with other computing systems (not shown) over communication networks (not shown). Examples of connections and devices that together allow for inter-system communication may include network interface cards, antennas, power amplifiers, RF circuitry, transceivers, and other communication circuitry. The connections and devices may communicate over communication media to exchange communications with other computing systems or networks of systems, such as metal, glass, air, or any other suitable communication media. The aforementioned media, connections, and devices are well known and need not be discussed at length here.

Communication between computing system 901 and other computing systems (not shown), may occur over a communication network or networks and in accordance with various communication protocols, combinations of protocols, or variations thereof. Examples include intranets, internets, the Internet, local area networks, wide area networks, wireless networks, wired networks, virtual networks, software defined networks, data center buses and backplanes, or any other type of network, combination of network, or variation thereof. The aforementioned communication networks and protocols are well known and need not be discussed at length here.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

The included descriptions and figures depict specific embodiments to teach those skilled in the art how to make and use the best mode. For the purpose of teaching inventive principles, some conventional aspects have been simplified or omitted. Those skilled in the art will appreciate variations from these embodiments that fall within the scope of the disclosure. Those skilled in the art will also appreciate that the features described above may be combined in many ways to form multiple embodiments. As a result, the invention is not limited to the specific embodiments described above, but only by the claims and their equivalents.

What is claimed is:

1. A contagion modeling system, comprising:
a first CO2 thermal depth imaging camera, having a first field of view;
a second CO2 thermal depth imaging camera, having a second field of view;
a processor, connected to the first and second CO2 thermal depth imaging cameras;
wherein the first and second CO2 thermal depth imaging cameras are configured so that the first and second fields of view are located within a modeling area, the modeling area comprising:
a first contagion source, where the contagion source is a gaseous fluid source;
a first stationary object; and
a first moving object.

2. The contagion modeling system of claim 1, wherein the first CO2 filtered thermal depth imaging camera comprises a first CO2 filtered thermal camera and a first depth camera, both configured to capture the first field of view.

3. The contagion modeling system of claim 1, wherein the processor is configured to translate first image data from the from the first CO2 thermal depth imaging camera into a first 3D density-flow representation.

4. The contagion modeling system of claim 3, wherein the processor is configured to translate second image data from the from the second CO2 thermal depth imaging camera into a second 3D density-flow representation.

5. The contagion modeling system of claim 4, wherein the first density-flow representation corresponds, at least in part, to the first contagion source.

6. The contagion modeling system of claim 3, wherein the processor is configured to calculate a predicted airflow pattern in the modeling area based at least on the first 3D density-flow representation, the first stationary object and the first moving object.

7. A method of modeling the spread of a contagion, comprising:
receiving first image data from a first CO2 filtered thermal camera, the image data corresponding to a first field of view within a modeling area, the modeling area comprising at least a gaseous flow contagion source, a stationary object and a moving object,
translating the first image data from the first CO2 thermal depth imaging camera into a first 3D density-flow representation;
processing the first 3D density-flow representation with at least the stationary object and the moving object to produce a predicted contagion flow pattern.

8. The contagion modeling method of claim 7, wherein the first CO2 filtered thermal depth imaging camera comprises a first CO2 filtered thermal camera and a first depth camera, both configured to capture the first field of view.

9. The contagion modeling method of claim 7, wherein the first density-flow representation corresponds, at least in part, to the first contagion source.

10. The contagion modeling method of claim 7, wherein the first image data is a set of progressive time-based image data.

11. A method of modeling a system to predict contagion dispersion, comprising:
acquiring a first thermal depth video stream;
identifying a first gaseous fluid source in the first thermal depth video stream, and converting the first gaseous fluid source in the first thermal depth video stream into a first fluid dispersion model;
identifying a second gaseous fluid source in the first thermal depth video stream, and converting the second gaseous fluid source in the first thermal depth video stream into a second fluid dispersion model;
determining that the first fluid dispersion model may interact with the second fluid dispersion model and a first stationary object, resulting in a density of the first fluid that is above a threshold value in a first location;
determining a recommended layout change in response to the determination that the density of the first fluid is above the threshold value in the first location; and
indicating the recommended layout change.

12. The method of claim 11, wherein acquiring the first thermal depth video stream comprises acquiring a video stream through a first CO2 filtered thermal camera and a first depth camera, both configured to capture a first field of view.

13. The method of claim 11, further comprising:
acquiring a second thermal depth video stream;
identifying the first gaseous fluid source in the second thermal depth video stream and converting the first gaseous fluid source in the second thermal depth video stream into a third fluid dispersion model; and
confirming the accuracy of the first fluid dispersion model with the third fluid dispersion model.

14. The method of claim 11, wherein the first fluid dispersion model corresponds, at least in part, to a first contagion source.

15. The method of claim 11, further comprising calculating a predicted airflow pattern of the first gaseous fluid source based at least on the first and second fluid dispersion models, the first stationary object and a first moving object.
